(19) United States
(12) Patent Application Publication
(10) Pub. No.: US 2025/0121164 A1
(43) Pub. Date: Apr. 17, 2025

(54) MULTI-DUROMETER POLYMER EXTRUSIONS FOR SMOOTH STIFFNESS TRANSITION IN INTRALUMINAL GUIDEWIRES AND/OR CATHETERS

(71) Applicant: KONINKLIJKE PHILIPS N.V., EINDHOVEN (NL)

(72) Inventor: Blake CORNELL, SAN DIEGO, CA (US)

(21) Appl. No.: 18/908,920

(22) Filed: Oct. 8, 2024

Related U.S. Application Data
(60) Provisional application No. 63/543,532, filed on Oct. 11, 2023.

(51) Int. Cl. A61M 25/09; A61L 31/00; A61L 31/10

(52) U.S. Cl. CPC A61M 25/09; A61L 31/005; A61L 31/10; A61M 2025/09083; A61M 2025/09133; A61M 2205/0238; A61M 2205/3303; A61M 2205/3327; A61M 2210/12

(57) ABSTRACT

An intravascular guidewire can be positioned within a blood vessel and includes a proximal portion and a distal portion, a core wire, a polymer jacket positioned around the core wire, a sensor housing positioned at the distal portion, and a sensor positioned within the rigid sensor housing that obtains medical data associated with the blood vessel. The polymer jacket can include a first section with a first hardness and a second section with a second hardness. The first section can be disposed directly adjacent to the sensor housing, and the first section can have a greater durometer than the second section. The first and second sections can be first and second compositions of at least two polymers. The polymer jacket can include a third section that is a transition between the first composition and the second composition.

### TECHNICAL FIELD

The subject matter described herein relates, in general, to intraluminal physiology sensing devices (e.g., an intravascular pressure sensing and/or flow sensing guidewire), and in particular to multi-durometer polymer extrusions for smooth transition between components with different stiffnesses.

### BACKGROUND

Heart disease is very serious and often requires emergency operations to save lives. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

One solution is to use intravascular devices such as catheters and guide wires to measure the pressure within the blood vessel, visualize the inner lumen of the blood vessel, and/or otherwise obtain data related to the blood vessel. Conventionally, intravascular catheters and guide wires include a stiff proximal portion that transitions into a soft distal portion. The proximal portion may be stiffer in order to maximize trackability and to allow for the intravascular device to be pushed. The distal portion is softer in order to prevent damage to the vasculature of a patient. The intravascular device can include a core member that is generally formed of an elastic and durable material, which allows the guide wire to traverse the tortuous anatomy, such as the patient's blood vessels. The core member generally extends along the length of the guide wire. The intravascular device often includes one or more rigid components disposed near the distal portion. The one or more components can include a housing inside which is positioned pressure sensors, flow sensor, and/or other obtain the data related to the blood vessel.

Due to the weight and/or shape of the one or more components, a structural weak point, kink point, or hinge point is located at the distal portion of the core member adjacent to the one or more components. These hinge points reduce mechanical performance and are weak spots for electrical and mechanical stress. Therefore, the hinge point may cause the intravascular device to unintentionally bend, kink, or break at the hinge point.

The information included in this Background section of the specification, including any references cited herein and any description or discussion thereof, is included for technical reference purposes only and is not to be regarded as subject matter by which the scope of the disclosure is to be bound.

### SUMMARY

The present disclosure provides an improvement for intraluminal devices (e.g., intravascular catheters and/or guidewires) by creating multi-durometer polymer extrusions (also referred to as polymer jackets) that enable a smooth transition and provide strain relief. Different combinations (or compositions) of two or more polymers are used along a length of an intraluminal device. In some situations, a harder polymer section is disposed more distal than a softer polymer section. For example, positioning the harder polymer section is immediately adjacent to a rigid housing provides for a smoother stiffness transition and helps avoids a kink point at this transition. In other situation, the softer polymer section is disposed more distal than the harder polymer section.

One general aspect includes an apparatus. The apparatus includes an intravascular guidewire configured to be positioned within a blood vessel and may include: a proximal portion and a distal portion; a core wire; a polymer jacket positioned around the core wire; a sensor housing positioned at the distal portion; and a sensor positioned within the sensor housing and configured to obtain medical data associated with the blood vessel. The polymer jacket may include a first section with a first hardness and a second section with a second hardness.

Implementations may include one or more of the following features. The first hardness is less than the second hardness. The first hardness is larger than the second hardness, where the first section is proximate to the sensor housing, and where the second section is proximate to the first section. The first section may include a first composition of at least two polymers, and where the second section may include a second composition of the at least two polymers. The polymer jacket may include a third section with a third hardness, where the third section is proximate to the second section such that the second section is disposed directly between the first section and the third section, and where the third section may include a third composition of the at least two polymers. The third hardness is larger than both the first hardness and the second hardness. The sensor housing may include a first proximal end and an opposing distal end, where the distal end is distal of the proximal end, and where the polymer jacket is in direct contact with the proximal end of the sensor housing. The third hardness is larger than the fourth hardness, where the fourth section is distal of the third section, and where the third section is proximate to the distal end of the sensor housing. The sensor housing may include a first proximal end and an opposing distal end, where the distal end is distal of the proximal end, where the polymer jacket is in direct contact with the proximal end of the sensor housing, and where the further polymer jacket is in direct contact with the distal end of the sensor housing.

One general aspect includes an apparatus. The apparatus includes an intravascular guidewire configured to be positioned within a blood vessel and may include: a proximal portion and a distal portion; a core wire; a polymer jacket positioned around the core wire; a sensor housing positioned at the distal portion; and a sensor positioned within the sensor housing and configured to obtain medical data associated with the blood vessel. The polymer jacket may include: a first section may include a first composition of at least two polymers; a second section may include a second composition of the at least two polymers; and a third section may include a third composition of the at least two polymers and located between the first section and the second section. The third composition may include a transition between the first composition and the second composition.

Implementations may include one or more of the following features. The first section may include a first hardness, and where the second section may include a second hardness different than first hardness. The first hardness is greater than the second hardness. The sensor housing is located at a distal end of the distal portion, and where the distal portion terminates at the distal end. The first section of the polymer jacket is in direct contact with the sensor housing. The first composition, the second composition, and the third composition each may include different percentages of the at least two polymers. The polymer jacket is disposed proximal of the sensor housing, and where the second section is proximal of the first section and proximal of the third section. The apparatus may include: a further polymer jacket disposed distal of the sensor housing and positioned around the core wire. The polymer jacket is disposed distal of the sensor housing, where the second section is distal of the third section, and where the third section is distal of the first section.

One general aspect includes an apparatus. The apparatus includes an intravascular guidewire configured to be positioned within a blood vessel and may include: a proximal portion and a distal portion; a core wire; a polymer jacket positioned around a length of the core wire; a rigid sensor housing positioned at the distal portion; and a sensor positioned within the rigid sensor housing and configured to obtain medical data associated with the blood vessel. The polymer jacket may include varying durometers along the length. The apparatus also includes where the polymer jacket may include a first section and a second section. The apparatus also includes where the first section is disposed directly proximate to the rigid sensor housing. The apparatus also includes where the first section has a greater durometer than the second section.

Implementations may include one or more of the following features. The second section is located proximal of the first section. The second section is located distal of the first section.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the multi-durometer polymer extrusion for smooth stiffness transition in guide wires, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic top view of an intravascular device, according to aspects of the present disclosure.

FIG. 2 is a diagrammatic side view of an intravascular sensing system that includes an intravascular device, according to aspects of the present disclosure.

FIG. 3A is a diagrammatic side view of a portion of an intravascular device in accordance with at least one aspect of the present disclosure.

FIG. 3B is a diagrammatic side view of the intravascular device of FIG. 3A, in accordance with at least one aspect of the present disclosure.

FIG. 3C is a diagrammatic side view of the intravascular device of FIG. 3B, in accordance with one or more aspects of the present disclosure.

FIG. 4 is a diagrammatic side view of an intravascular device with two multi-durometer polymer jackets, in accordance with at least one aspect of the present disclosure.

FIG. 5 is another diagrammatic side view of the intravascular device with a multi-durometer polymer jacket surrounding a core member having a constant diameter, in accordance with at least one aspect of the present disclosure.

FIG. 6 is yet another diagrammatic side view of the intravascular device with a multi-durometer polymer jacket surrounding a core member having varying diameters, in accordance with at least one aspect of the present disclosure.

FIG. 7 is yet another diagrammatic side view of a distal tip portion of the intravascular device with a multi-durometer polymer jacket disposed at the distal tip portion, in accordance with at least one aspect of the present disclosure.

### DETAILED DESCRIPTION

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. Further, while the aspects of the present disclosure may be described with respect to a blood vessel, it will be understood that the devices, systems, and methods described herein may be configured for use in any suitable anatomical structure or body lumen including a blood vessel, blood vessel lumen, an esophagus, eustachian tube, urethra, fallopian tube, intestine, colon, and/or any other suitable anatomical structure or body lumen. In other aspects, the devices, systems, and methods described herein may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood vessels, chambers or other parts of the heart, and/or other systems of the body. In addition to natural structures, the intraluminal devices, described herein, may be used to examine man-made structures such as, but without limitation, heart valves, stents, shunts, filters, and other devices. For the sake of brevity, however, the numerous iterations of these combinations will not be described separately.

The example aspects described below recognize that it may be desirable to have a method of manufacturing that includes extruding at least two polymers having two, different durometer values to form at least one polymer jacket(s). In particular, the aspects described below provide a method for extruding a first polymer with a first durometer value and extruding a second polymer with a second, differing durometer value at various ratios along a length of an intravascular device (such as a guide wire) to create a multi-durometer polymer jacket. The method of extrusion provides a way to create a multi-durometer polymer jacket. In particular, the method allows for extruding a first polymer composition (of the first polymer and the second polymer) with a greater durometer value at a structurally weak “hinge” point (such as where the core member couples to the housing), extruding a second polymer composition that has a lesser durometer value in comparison to the first polymer composition next to the first polymer composition, extruding a third polymer composition that has a greater durometer value in comparison to the second polymer composition near the proximal portion of the intravascular device, and etc. Therefore, the method of manufacturing provides an improvement by creating a multi-durometer polymer jacket with a greater durometer value near the hinge points.

One or more aspects described below provide apparatuses related to forming multi-durometer jackets for an intravascular device. For example, in some instances, a method is provided for extruding a first polymer with a first durometer value along a first length of an intravascular device, extruding at least one polymer composition that includes the first polymer and a second polymer along a second length adjacent to the first length, and extruding the second polymer with a second durometer value along a third length of the intravascular device adjacent to the second length. The first durometer value is greater than the durometer value of the polymer composition and is greater than the durometer value of the second polymer. The first polymer is adjacent to a hinge point of the intravascular device. Therefore, the method of manufacturing provides an improvement by changing the material properties of the polymer jacket rather than the geometry of the polymer jacket in order to reduce mechanical and electrical strain.

One or more illustrative aspects described below provide apparatuses related to multi-durometer polymer jackets for an intravascular device. A proximal portion of the intravascular device must be stiff enough to traverse (or be pushed through) the tortuous anatomy of a patient's vasculature; however, the distal portion of the intravascular device must be flexible enough to not damage the patient's vasculature. However, the intravascular device may include a hinge point (location prone to bending/kinking), this hinge point may need to include strain relief to prevent this unintended bending. These methods and apparatuses provide at least one multi-durometer polymer jacket that increases stiffness at segments near hinge points (such as locations near the housing/sensors) and reduces stiffness at segments of the polymer jacket adjacent to the segments near hinge points.

Further, one or more illustrative aspects described below provide methods for creating smooth stiffness transitions and apparatuses of multi-durometer polymer jackets. For example, placing a segment of one polymer directly adjacent to a segment of another polymer may create another structurally weak spot of the intravascular device. Therefore, by extruding a first polymer and a second polymer at varying ratios along a length of the intravascular device allows for smooth stiffness transitions. By creating smooth stiffness transitions, additional structural weak points are not added. Instead, by creating smooth stiffness transitions within the polymer jacket, the polymer jacket may help prevent other structural weak spots of the intravascular device.

Further still, one or more illustrative aspects described below provide methods for improving the support profile or the bending moment of the intravascular device. For example, in some instances, the intravascular device may include a core wire with a tapering tip at a distal end of the intravascular device. The tapering tip has varying diameters. The tapering tip may have portions with constant slope and portions with increasing (or even infinite/indefinite slope). In various aspects, a polymer or polymer composition with a higher durometer value is disposed at areas of the tapering tip with increasing (or infinite/indefinite slope) and have a differing polymer or polymer composition with a lower durometer value disposed at areas of the tapering tip with constant slope, which will produce a more linear support profile for the intravascular device. The methods and apparatuses described herein may produce an improved support profile for the intravascular device.

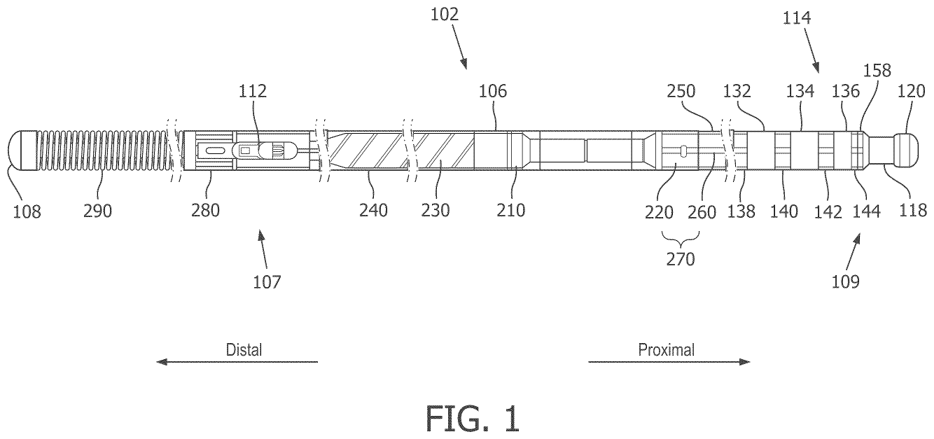

FIG. 1 is a diagrammatic top view of an intravascular device 102, according to aspects of the present disclosure. The intravascular device 102 may be an intravascular, intraluminal, or endoluminal guidewire, catheter, or guide catheter sized and shaped for positioning within a blood vessel of a patient. The intravascular device 102 may include a sensor 112. For example, the sensor 112 may be a pressure sensor configured to measure a pressure of blood flow within the vessel of the patient. The intravascular device 102 includes the flexible elongate member 106. The sensor 112 is disposed at the distal portion 107 of the flexible elongate member 106. The sensor 112 may be mounted at the distal portion 107 within a housing 280 in some aspects. A flexible tip coil 290 extends between the housing 280 and the distal end 108. The connection portion 114 is disposed at the proximal portion 109 of the flexible elongate member 106. The connection portion includes the conductive portions 132, 134, 136. In various aspects, the conductive portions 132, 134, 136 may be conductive ink that is printed and/or deposited around the flexible elongate member 106. In some aspects, the conductive portions 132, 134, 136 may be conductive, metallic rings that are positioned around the flexible elongate member. The locking section 118 and knob or retention section 120 are disposed at the proximal portion 109 of the flexible elongate member 106.

The intravascular device 102 in FIG. 1 includes a distal core 210 and a proximal core 220. The distal core 210 and the proximal core 220 are metallic components forming part of the body of the intravascular device 102. For example, the distal core 210 and the proximal core 220 are flexible metallic rods that provide structure for the flexible elongate member 106. The diameter of the distal core 210 and the proximal core 220 may vary along its length.

In some various, the intravascular device 102 comprises a distal assembly and a proximal assembly that are electrically and mechanically joined together, which results in electrical communication between the sensor 112 and the conductive portions 132, 134, 136. For example, pressure data obtained by the sensor 112 (in this example, sensor 112 is a pressure sensor) may be transmitted to the conductive portions 132, 134, 136. Control signals from a computer in communication with the intravascular device 102 may be transmitted to the sensor 112 via the conductive portions 132, 134, 136. The distal subassembly may include the distal core 210. The distal subassembly may also include the sensor 112, conductive members 230, and/or one or more layers of polymer/plastic 240 surrounding the conductive members 230 and the core 210. For example, the polymer/plastic layer(s) may protect the conductive members 230. The proximal subassembly may include the proximal core 220. The proximal subassembly may also include one or more layers of polymer layer(s) 250 (hereinafter polymer layer 250) surrounding the proximal core 220 and/or conductive ribbons 260 embedded within the one or more layers of polymer layer(s) 250. In some aspects, the proximal subassembly and the distal subassembly may be separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly 210 (e.g., including the distal core 210, etc.).

In various aspects, the intravascular device 102 may include one, two, three, or more core wires extending along its length. For example, a single core wire may extend substantially along the entire length of the flexible elongate member 106. In such aspects, the locking section 118 and the knob or retention section 120 may be integrally formed at the proximal portion of the single core wire. The sensor 112 may be secured at the distal portion of the single core wire. In other aspects, such as the aspect illustrated in FIG. 1, the locking section 118 and the knob or retention section 120 may be integrally formed at the proximal portion of the proximal core 220. The sensor 112 may be secured at the distal portion of the distal core 210. The intravascular device 102 includes one or more conductive members 230 in communication with the electronic component (sensor 112). For example, the conductive members 230 may be one or more electrical wires that are directly in communication with the sensor 112. In some instances, the conductive members 230 are electrically and mechanically coupled to the sensor 112 by, e.g., soldering. In some instances, the conductive members 230 comprise two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210.

The intravascular device 102 includes one or more conductive ribbons 260 at the proximal portion of the flexible elongate member 106. The conductive ribbons 260 are embedded within polymer layer(s) 250. The conductive ribbons 260 are directly in communication with the conductive portions 132, 134, and/or 136. In some instances, the conductive members 230 are electrically and mechanically coupled to the sensor 112 by, e.g., soldering. In some instances, the conductive portions 132, 134, and/or 136 comprise conductive ink (e.g., metallic nano-ink, such as silver or gold nano-ink) that is deposited or printed directed over the conductive ribbons 260.

As described herein, electrical communication between the conductive members 230 and the conductive ribbons 260 may be established at the connection region 270 of the flexible elongate member 106. By establishing electrical communication between the conductive members 230 and the conductive ribbons 260, the conductive portions 132, 134, 136 may be in electrically communication with the sensor 112.

In some instances, represented by FIG. 1, intravascular device 102 includes the locking section 118 and the knob or retention section 120. To form the locking section 118, a machining process is necessary to remove the polymer layer 250 and the conductive ribbons 260 in the locking section 118 and to shape proximal core 220 in the locking section 118 to the desired shape. As shown in FIG. 1, the locking section 118 includes a reduced diameter while the knob or retention section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons.

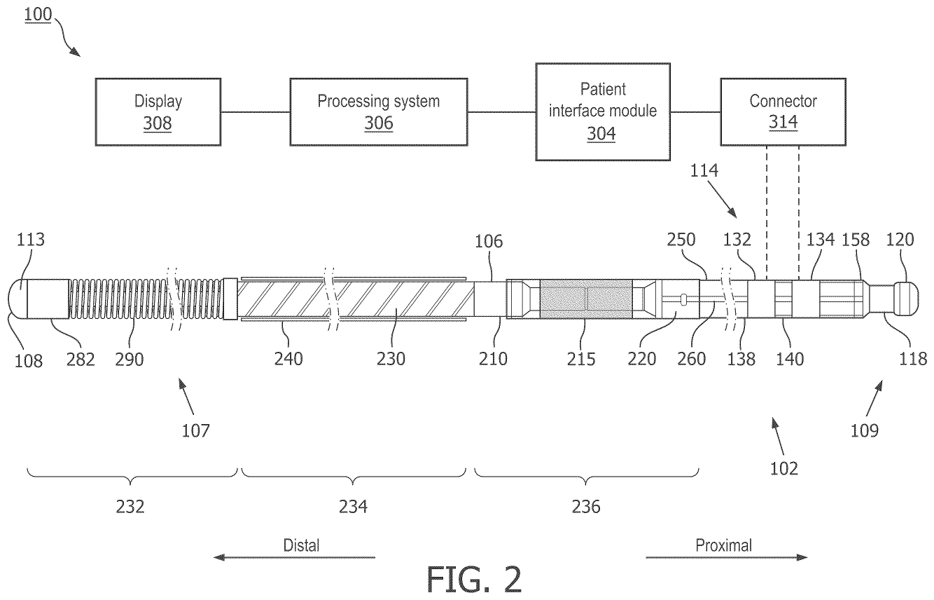

FIG. 2 is a diagrammatic side view of an intraluminal (e.g., intravascular) sensing system 100 that includes an intravascular device 102 comprising conductive members 230 (e.g., a multi-filar electrical conductor bundle) and conductive ribbons 260, according to aspects of the present disclosure. The intravascular device 102 may be an intravascular guidewire sized and shaped for positioning within a blood vessel of a patient. The intravascular device 102 includes a distal tip 108 and a sensor 113. For example, the sensor 113 may be a pressure sensor and/or flow sensor configured to measure a pressure of blood flow within the vessel of the patient, or another type of sensor including but not limited to a temperature or imaging sensor, or combination sensor measuring more than one property. For example, the flow data obtained by a flow sensor may be used to calculate physiological variables such as coronary flow reserve (CFR). The intravascular device 102 includes a flexible elongate member 106. The sensor 113 is disposed at a distal portion 107 of the flexible elongate member 106. The sensor 113 may be mounted at the distal portion 107 within a housing 282 in some aspects. A flexible tip coil 290 extends distally from the housing 282 at the distal portion 107 of the flexible elongate member 106. A connection portion 114 located at a proximal end of the flexible elongate member 106 includes conductive portions 132, 134. In some aspects, the conductive portions 132, 134 may be conductive ink that is printed and/or deposited around the connection portion 114 of the flexible elongate member 106. In some aspects, the conductive portions 132, 134 are conductive, may be metallic bands or rings that are positioned around the flexible elongate member. A locking area is formed by a collar or locking section 118 and knob or retention section 120 are disposed at the proximal portion 109 of the flexible elongate member 106.

The intravascular device 102 in FIG. 2 includes core wire comprising a distal core 210 and a proximal core 220. The distal core 210 and the proximal core 220 are metallic components forming part of the body of the intravascular device 102. For example, the distal core 210 and the proximal core 220 may be flexible metallic rods that provide structure for the flexible elongate member 106. The distal core 210 and/or the proximal core 220 may be made of a metal or metal alloy. For example, the distal core 210 and/or the proximal core 220 may be made of stainless steel, Nitinol, nickel-cobalt-chromium-molybdenum alloy (e.g., MP35N), and/or other suitable materials. In some aspects, the distal core 210 and the proximal core 220 are made of the same material. In other aspects, the distal core 210 and the proximal core 220 are made of different materials. The diameter of the distal core 210 and the proximal core 220 may vary along their respective lengths. A joint between the distal core 210 and proximal core 220 is surrounded and contained by a hypotube 215. The sensor 113 may in some cases be positioned at a distal end of the distal core 210.

In some aspects, the intravascular device 102 comprises a distal subassembly and a proximal subassembly that are electrically and mechanically joined together, which creates an electrical communication between the sensor 113 and the conductive portions 132, 134. For example, flow data obtained by the sensor 113 (in this example, sensor 113 is a flow sensor) may be transmitted to the conductive portions 132, 134. In an exemplary aspect, the sensor 113 is a single ultrasound transducer element. The transducer element emits ultrasound signals and receives echoes. The transducer element generates electrical signals representative of the echoes. The signal carrying filars carry this electrical signal from the sensor at the distal portion to the connector at the proximal portion. The processing system 306 processes the electrical signals to extract the flow velocity of the fluid.

Control signals from a processing system 306 (e.g., a processor circuit of the processing system 306) in communication with the intravascular device 102 may be transmitted to the sensor 113 via a connector 314 that is attached to the conductive portions 132, 134. The distal subassembly may include the distal core 210. The distal subassembly may also include the sensor 113, the conductive members 230, and/or one or more layers of insulative polymer/plastic 240 surrounding the conductive members 230 and the core 210. For example, the polymer/plastic layer(s) may insulate and protect the conductive members of the multi-filar cable or conductor bundle 230. The proximal subassembly may include the proximal core 220. The proximal subassembly may also include one or more polymer layers 250 (hereinafter polymer layer 250) surrounding the proximal core 220 and/or conductive ribbons 260 embedded within the one or more insulative and/or protective polymer layer 250. In some aspects, the proximal subassembly and the distal subassembly are separately manufactured. During the assembly process for the intravascular device 102, the proximal subassembly and the distal subassembly may be electrically and mechanically joined together. As used herein, flexible elongate member 106 may refer to one or more components along the entire length of the intravascular device 102, one or more components of the proximal subassembly (e.g., including the proximal core 220, etc.), and/or one or more components the distal subassembly (e.g., including the distal core 210, etc.). Accordingly, flexible elongate member 106 may refer to the combined proximal and distal subassemblies described above. The joint between the proximal core 220 and distal core 210 is surrounded by the hypotube 215.

In various aspects, the intravascular device 102 may include one, two, three, or more core wires extending along its length. For example, a single core wire may extend substantially along the entire length of the flexible elongate member 106. In such aspects, a locking section 118 and a section 120 may be integrally formed at the proximal portion of the single core wire. The sensor 113 may be secured at the distal portion of the single core wire. In other aspects, such as the aspect illustrated in FIG. 2, the locking section 118 and the section 120 may be integrally formed at the proximal portion of the proximal core 220. The sensor 113 may be secured at the distal portion of the distal core 210. The intravascular device 102 includes one or more conductive members 230 (e.g., a multi-filar conductor bundle or cable) in communication with the sensor 113. For example, the conductive members 230 may be one or more electrical wires that are directly in communication with the sensor 113. In some instances, the conductive members 230 are electrically and mechanically coupled to the sensor 113 by, e.g., soldering. In some instances, the conductor bundle 230 comprises two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210, minimizing or eliminating whipping of the distal core within tortuous anatomy.

The intravascular device 102 includes one or more conductive ribbons 260 at the proximal portion of the flexible elongate member 106. The conductive ribbons 260 are embedded within polymer layer 250. The conductive ribbons 260 are directly in communication with the conductive portions 132 and/or 134. In some instances, a multi-filar conductor bundle 230 is electrically and mechanically coupled to the sensor 113 by, e.g., soldering. In some instances, the conductive portions 132 and/or 134 comprise conductive ink (e.g., metallic nano-ink, such as copper, silver, gold, or aluminum nano-ink) that is deposited or printed directed over the conductive ribbons 260.

As described herein, electrical communication between the conductive members 230 and the conductive ribbons 260 may be established at the connection portion 114 of the flexible elongate member 106. By establishing electrical communication between the conductor bundle 230 and the conductive ribbons 260, the conductive portions 132, 134 may be in electrical communication with the sensor 113.

In some aspects represented by FIG. 1, the intravascular device 102 includes a locking section 118 and knob or retention section 120. To form locking section 118, a machining process is used to remove polymer layer 250 and conductive ribbons 260 in locking section 118 and to shape proximal core 220 in locking section 118 to the desired shape. As shown in FIG. 1, locking section 118 includes a reduced diameter while knob or retention has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons 260.

In some aspects, a connector 314 provides electrical connectivity between the conductive portions 132, 134 and a patient interface monitor 304. The Patient Interface Monitor (PIM) 304 may in some cases connect to a console or processing system 306, which includes or is in communication with a display 308.

The system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some aspects, device 102 may be controlled from a remote location such as the control room, such that an operator is not required to be in close proximity to the patient.

The intraluminal device 102, PIM 304, and display 308 may be communicatively coupled directly or indirectly to the processing system 306. These elements may be communicatively coupled to the medical processing system 306 via a wired connection such as a standard copper multi-filar conductor bundle 230. The processing system 306 may be communicatively coupled to one or more data networks, e.g., a TCP/IP-based local area network (LAN). In other aspects, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed (e.g., as physiology data in graphical, symbolic, or alphanumeric form) on the display 308. The console or processing system 306 may include a processor and a memory. The processing system 306 may be operable to facilitate the features of the intravascular sensing system 100 described herein. For example, the processor may execute computer readable instructions stored on the non-transitory tangible computer readable medium.

The PIM 304 facilitates communication of signals between the processing system 306 and the intraluminal device 102. The PIM 304 may be communicatively positioned between the processing system 306 and the intraluminal device 102. In some aspects, the PIM 304 performs preliminary processing of data prior to relaying the data to the processing system 306. In examples of such aspects, the PIM 304 performs amplification, filtering, and/or aggregating of the data. In an aspect, the PIM 304 also supplies high- and low-voltage DC power to support operation of the intraluminal device 102 via the conductive members 230.

A multi-filar cable or transmission line bundle 230 may include a plurality of conductors, including one, two, three, four, five, six, seven, or more conductors. In the example shown in FIG. 2, the multi-filar conductor bundle 230 includes two straight portions 232 and 236, where the multi-filar conductor bundle 230 lies parallel to a longitudinal axis of the flexible elongate member 106, and a spiral portion 234, where the multi-filar conductor bundle 230 is wrapped around the exterior of the flexible elongate member 106 and then overcoated with an insulative and/or protective polymer 240. Communication, if any, along the multi-filar conductor bundle 230 may be through numerous methods or protocols, including serial, parallel, and otherwise, where one or more filars of the bundle 230 carry signals. One or more filars of the multi-filar conductor bundle 230 may also carry direct current (DC) power, alternating current (AC) power, or serve as a ground connection.

The display or monitor 308 may be a display device such as a computer monitor or other type of screen. The display or monitor 308 may be used to display selectable prompts, instructions, and visualizations of imaging data to a user. In some aspects, the display 308 may be used to provide a procedure-specific workflow to a user to complete an intraluminal imaging procedure.

Before continuing, it should be noted that the examples described above are provided for purposes of illustration and are not intended to be limiting. Other devices and/or device configurations may be utilized to carry out the operations described herein.

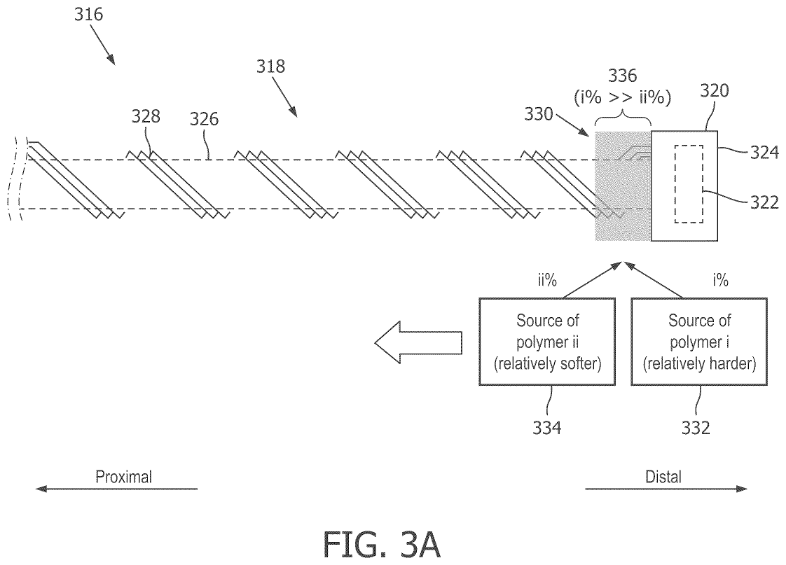

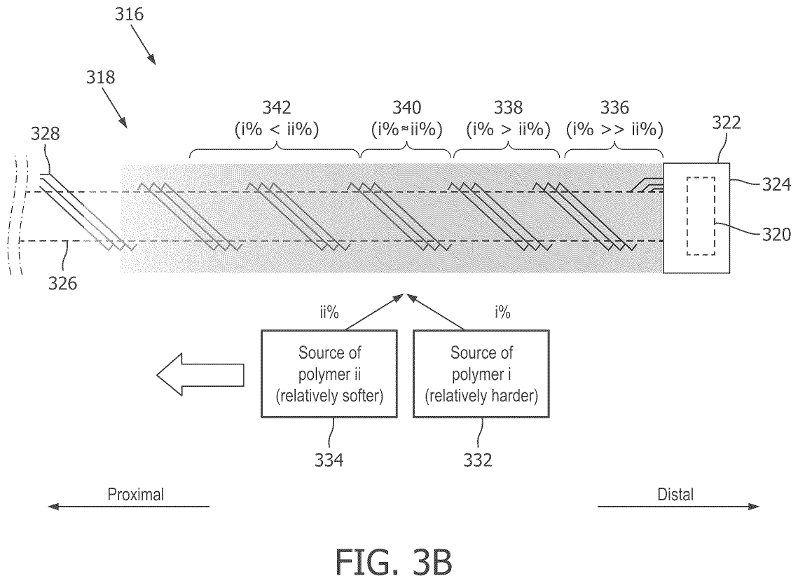

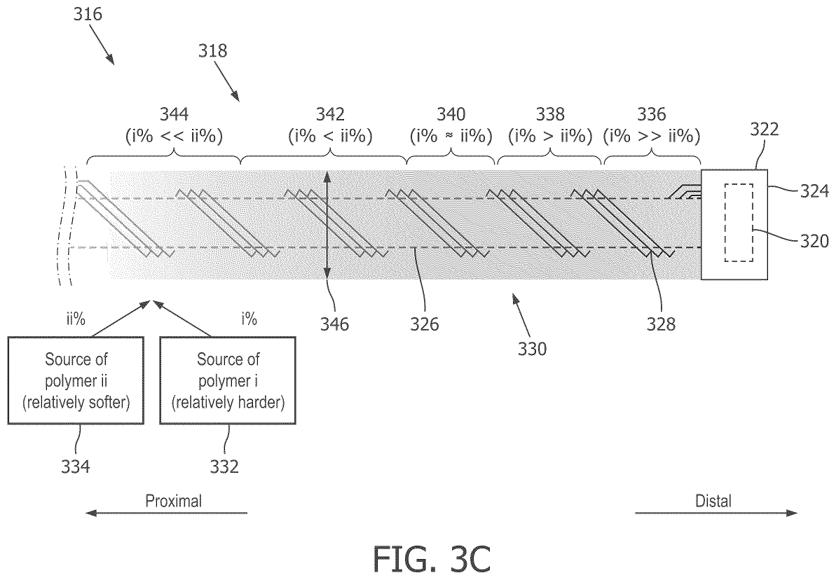

Collectively, FIGS. 3A-3C illustrate aspects of a multi-durometer polymer jacket for an intravascular device according to the present disclosure. In particular, FIGS. 3A-3C illustrate the multi-durometer polymer jacket of the intravascular device at different stages of manufacturing according to one or more aspects. In some instances, FIGS. 3A-3C may illustrate a distal portion of an intraluminal device (e.g., intravascular guidewire), such as the distal portion 107 of the pressure-sensing guidewire (intravascular device 102) in FIG. 1, and/or the distal portion 107 of the flow sensing guidewire (intravascular device 102) in FIG. 2.

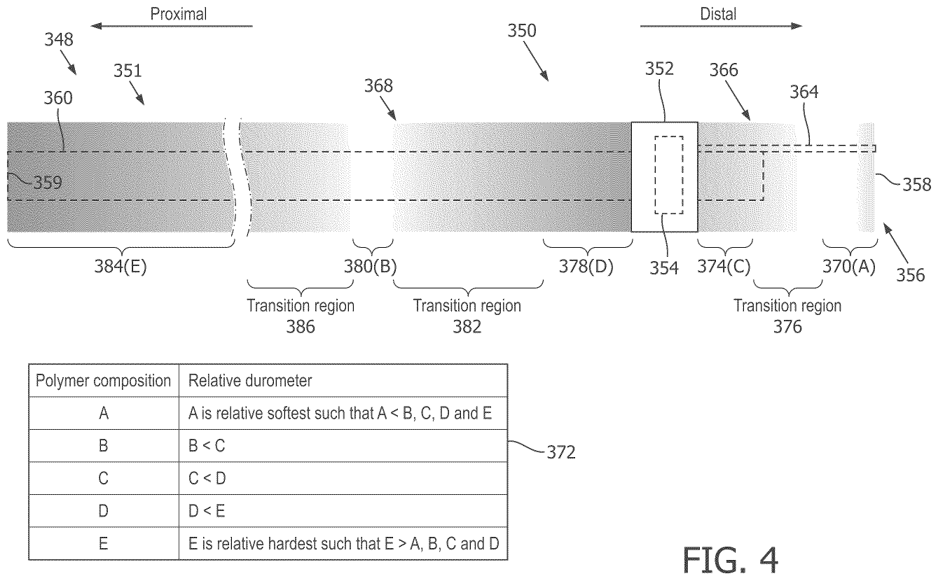

Referring to FIG. 3A, shown therein is a diagrammatic, schematic side view of a distal portion 318 of an intraluminal device 316 (e.g., a guidewire, such as an intravascular guidewire, or a catheter, such as an intravascular catheter or an intracardiac catheter) according to an aspect of the present disclosure. As shown, the distal portion 318 includes a housing 320 containing component 322. The component 322 is representative of one or more electronic, optical, or electro-optical components. The housing 320 includes a distal tip 324 defining an end to the distal portion 318. A core member 326 extends laterally from the housing 320 and communication lines 328 extend from the housing 320 along a length of the core member 326. A polymer jacket 330 is disposed adjacent to the housing 320 and includes at least two polymers: polymer i 332 and polymer ii 334. Polymer i 332 is comprised of polymer that is relatively harder than polymer ii 334, which is relatively softer than polymer i 332. The polymer jacket 330 includes a first segment 336. The first segment 336 is formed around the core member 326 and disposed in direct contact with the housing 320. The first segment 336 may include a composition of at least: polymer i 332 and polymer ii 334. The first segment 336 includes a percentage of polymer i 332 that is far greater than the percentage of polymer ii 334. Exemplary numerical ranges are provided in the description of FIG. 4.

Aspects of the present disclosure may include features described in U.S. Pat. No. 11,219,748 filed Apr. 13, 2016, entitled “Intravascular Devices, Systems, and Methods Having a Polymer Jacket Formed Around Communication Lines Wrapped Around a Core Member,” the entirety of which is hereby incorporated by reference herein.

The core member 326 can be a metal or a metal alloy. The core member 326 can be a support member or structural backbone, providing structural support along the length of the intraluminal device 316. The core member 326 can provide a foundation for the application of the polymer jacket 330. In some instances, the core member 326 has a solid cross-section (e.g., no lumen). In other instances, the core member 326 can also be a hypotube or a hollow tube construction that does not have a solid cross-section (e.g., a wall of the hypotube or hollow tube defines a lumen).

Referring to FIG. 3B, with continuing reference to FIG. 3A, another aspect of the distal portion 318 of the intraluminal device 316 is shown and includes several components of FIG. 3A, which components are given the same reference number. The polymer jacket 330 includes the first segment 336, a second segment 338, a third segment 340, and a fourth segment 342. The first, second, third, and fourth segments 336, 338, 340, and 342, respectively, each comprise a different composition and/or ratio of polymer i 332 and polymer ii 334. The first segment 336 is directly adjacent to the housing 320 and the second segment 338. The third segment 340 is disposed between the second segment 338 and the fourth segment 342. The first segment 336 comprises the greatest percentage of polymer i 332 and the smallest percentage of the polymer ii 334 of the polymer jacket 330 such that the ratio of polymer i 332 is far greater than that of polymer ii 334. The second segment 338 also includes a composition of polymer i 332 and polymer ii 334. However, in the second segment 338, the ratio of polymer i 332 to polymer ii 334 is less than the first segment 336. The third segment 340 also includes a composition including at least the polymer i 332 and polymer ii 334 at equal or substantially equal amounts such that the ratio of polymer i 332 and polymer ii 334 is approximately 1:1. The fourth segment 342 also includes a composition of polymer i 332 and polymer ii 334. The fourth segment 342 comprises the smallest percentage of polymer i 332 of the polymer jacket 330, and the greatest percentage of the polymer ii 334 in comparison to the first, second, and third segments 336, 338, and 340, respectively.

Referring to FIG. 3C, with continuing reference to FIGS. 3A and 3B, another aspect of the distal portion 318 of the intraluminal device 316 is shown and includes several components of FIGS. 3A and 3B, which components are given the same reference number. The polymer jacket 330 includes the first segment 336, the second segment 338, the third segment 340, the fourth segment 342, and the fifth segment 344. The first, second, third, fourth, and fifth segments 336, 338, 340, 342, and 344, respectively, each comprise a different composition of polymer i 332 and polymer ii 334. The first, second, third, and fourth segments 336, 338, 340, and 342 are described in FIGS. 3A-3B. The fifth segment 344 also includes a composition of polymer i 332 and polymer ii 334. The fifth segment 344 comprises the smallest percentage of polymer i 332 of the polymer jacket 330, and the greatest percentage of the polymer ii 334 in comparison to the first, second, third, and fourth segments 336, 338, 340 and 342, respectively. The polymer jacket 330 further includes an outer diameter 346.

The polymer jacket 330, as shown in FIG. 3C, can be formed to have a substantially uniform outer diameter 346 despite the lack of uniformity in diameter resulting from wrapping the communication lines 328 around the core member 326. Generally, the outer diameter 346 is approximately equal to the maximum desired outer diameter of the intraluminal device 316.

With continuing reference to FIGS. 3A-C, the distal portion 318 of the intraluminal device 316 may be formed using any suitable approach so long as the polymer jacket 330 is formed around the communication lines and core in accordance with the present disclosure. In some implementations the intraluminal device 316 includes features similar to the distal, intermediate, and/or proximal sections described in one or more of U.S. Pat. Nos. 5,125,137, 5,873,835, 6,106,476, 6,551,250, U.S. patent application Ser. No. 13/931,052, filed Jun. 28, 2013, U.S. patent application Ser. No. 14/135,117, filed Dec. 19, 2013, U.S. patent application Ser. No. 14/137,364, filed Dec. 20, 2013, U.S. patent application Ser. No. 14/139,543, filed Dec. 23, 2013, U.S. patent application Ser. No. 14/143,304, filed Dec. 30, 2013, and U.S. Provisional Patent Application No. 61/935,113, filed Feb. 3, 2014, each of which is hereby incorporated by reference in its entirety.

The housing 320 can be made of a rigid material. The rigid material, in some instances, may be a metal or metal alloy (e.g., stainless steel), a relatively higher durometer polymer, and the like. In some instances, the housing 320 can be relatively more rigid (e.g., have a larger durometer hardness) than any polymer or polymer composition/combination forming the polymer jacket 330. In other instances, the housing 320 can have a similar hardness to one or more of the polymers or polymer compositions/combinations forming the polymer jacket 330. For example, the hardness of polymer or polymer composition/combination of the polymer jacket that is adjacent to, in contact with, and/or otherwise proximate to the housing 320 can be selected such that there is a smooth stiffness transition therebetween.

In various instances, the component 322 is a pressure sensor, a flow sensor, a temperature sensor, an imaging element, an optical fiber, an ultrasound transducer, a reflector, a mirror, a prism, an ablation element, an RF electrode, a conductor, and/or combinations thereof. The specific type of component or combination of components can be selected based on an intended use of the intravascular device. In some instances, the component 322 is positioned within the housing 320. In one or more instances, the component 322 is positioned on a sensor mount inside the housing 320. In some instances, between one and ten electrical conductors and/or optical pathways (e.g., communication lines 328) extend along the length of the core member 326 between a connector (not shown in FIGS. 3A-3C) and the component 322. The number of communication pathways and the number of electrical conductors and optical pathways (e.g., communication lines 328) extending is determined by the desired functionality of the component 322 and the corresponding elements that define component 322 to provide such functionality. In some instances, the polymer jacket 330 is coupled to the component 322. In one or more aspects, the polymer jacket 330 is coupled to a sensor mount inside the housing 320. In some instances, the polymer jacket 330 may be in direct contact with the housing 320 and/or the sensor mount. In other aspects, an adhesive may provide contact and/or coupling between the polymer jacket 330 and the housing 320 and/or the sensor mount.

Generally, the core member 326 is sized, shaped, and/or formed out of particular material(s) to create a desired mechanical performance for the distal portion 318 of the intraluminal device 316.

In some aspects, the communication lines 328 are wrapped around the core member 326 as shown in FIG. 3A-3C. However, in other aspects, communication lines 328 may extend linearly (e.g., extend parallel to the core member 326) or an alternative configuration along the length of the core member 326. In one or more aspects, the polymer jacket 330 is formed directly around the core member 326 and communication lines 328, which may be wrapped around the core member 326. FIG. 3C provides an image of a distal portion 318 of an intraluminal device 316 showing the polymer jacket 330 formed around communication lines 328 that are spiral wrapped around the core member 326. In the illustrated aspect, three communication lines 328 are shown. In other aspects, a different number of communication lines 328 are used.

In some instances, the polymer jacket 330 extends the entire length of the core member 326 positioned proximal to the component 322. In this regard, in some instances the polymer jacket 330 will abut a corresponding outer member of a flexible elongate member or other component of a guide wire, which will typically have the same outer diameter as the polymer jacket 330.

The polymer jacket 330 protects the communication lines 328 from damage during use, while maintaining flexibility of the intraluminal device 316. Additionally, the polymer jacket 330 prevents a twisting and/or break point near the housing 320 by placing a more rigid composition of polymers (e.g., the first segment 336) in between the housing 320 and a more flexible segment (e.g., the second segment 338). Therefore, the polymer i 332 and the polymer ii 334 utilized must be durable and biocompatible. Accordingly, the polymer i 332 can be any suitable material or combination of materials, including without limitation pellethane, other polyurethanes, nylon, pebax or other thermoplastics of suitable flexibility, and/or combinations thereof, and polymer ii 334 can be any suitable material or combination of materials, including without limitation pellethane, other polyurethanes, nylon, pebax or other thermoplastics of suitable flexibility, and/or combinations thereof. In some aspects, polymer i 332 and polymer ii 334 are a different material and/or combination of materials. Also, in some aspects the polymer jacket 330 is coated with a lubricious hydrophilic coating. In some aspects, a plurality of polymers are used for the polymer jacket 330 are used (e.g., three, four, or five polymers) each with different levels of hardness (i.e., durometer value).

In some aspects, more segments of the polymer jacket 330 are included along the length of the distal portion 318. In one or more aspects, less segments are included in forming the polymer jacket 330. In one or more aspects, the first, second, third, fourth, and/or fifth segments 336, 338, 340, 342, and 344 may have a different length along the core member 326 and/or distal portion 318. In various aspects, the first segment 336 is the shortest segment along the length. In one or more aspects, the first segment 336 includes only one polymer such as polymer i 332. In at least one aspect, the fifth segment 344 is the longest segment along the length. In one or more aspects, the fifth segment 344 includes only one polymer such as polymer ii 334. In some aspects, the second segment 338 and the fourth segment 342 are considered transition regions. In some aspects, the first segment 336 includes no polymer ii 334. In one or more aspects, the fifth segment 344 includes no polymer i 332. In other aspects, one of the transition regions (such as the second segment 338 or the fourth segment 342) is the shortest segment of the polymer jacket 330 along the length. In various instances, all, most, or some of the segments including the first, second, third, fourth, and/or fifth segments 336, 338, 340, 342, and 344 are a combination of two or more polymers (polymer i 332 and polymer ii 334). The multi-durometer polymer jacket 330 may be used at any suitable location of the intraluminal device 316 (e.g., the proximal portion, the distal portion, and the like.) In some instances, the multi-durometer polymer jacket 330 can form the structure of the body of the intravascular device 316 such as a guidewire or a catheter. For example, for a catheter, the multi-durometer polymer jacket 330 may define a lumen (e.g., a guidewire lumen or a lumen for communication lines, such as electrical wires, carrying signals from component 322 inside housing 320). In some aspects, the polymer jacket 330 includes compositions of two, three, four, or more polymers. In one or more aspects, compositions may also be referred to as a combination. In several aspects, each polymer in the polymer jacket 330 may itself be a combination (or composition) of multiple material sub-units (e.g., that are repeating). The combination (or combination) used for the polymer jacket 330 may be a combination of multiple polymers themselves (combination of combinations of multiple material sub-units).

Accordingly, in some particular implementations, the outer diameter 346 is about 0.014″, 0.018″, or 0.035″ (e.g., for an intravascular guidewire). In other implementations, such as for an intravascular catheter (e.g., an intravascular ultrasound or IVUS imaging catheter) or intracardiac catheter (e.g., an intracardiac echocardiography or ICE catheter), the outer diameter can be between 2.5 Fr and 9 Fr, between 2.5 Fr and 4 Fr, etc., and/or other ranges, including values such as 3 Fr, 3.3 Fr, 3.4 Fr, 3.5 Fr, 8.2 Fr, and/or other values both larger and smaller. In some implementations, the outer diameter 346 is achieved by passing the core member 326 with the polymer applied thereto through an opening having the desired outer diameter, machining/laser ablating/cutting or otherwise processing the applied polymer to achieve the desired outer diameter, and/or combinations thereof.

A method of forming or manufacturing the polymer jacket 330 that is multi-durometer value, in some aspects, includes extruding the first segment 336, which is a polymer composition, over the core member 325 with communication lines 328 wound around the core member 326 (as shown in FIG. 3A). The first segment 336 is formed such that more of polymer i 332 is extruded than polymer ii 334 in the first segment 336. The first segment 336 begins directly adjacent to and in direct contact with the housing 320. Next, the second segment 338 through a fourth segment 342 are extruded (as shown in FIG. 3B). The second segment 338 is formed directly adjacent to and in direct contact with the first segment 336 such that the polymer composition of the second segment 338 includes less of polymer i 332 and more of polymer ii 334 in comparison to the first segment 336. The third segment 340 is then formed directly adjacent to and in direct contact with the second segment 338 such that the polymer composition of the third segment 340 is approximately equal portions of polymer i 332 and polymer ii 334. Then the fourth segment 342 is formed directly adjacent to and in direct contact with the third segment 340 such that the polymer composition of the fourth segment 342 includes a greater portion of polymer ii 334 than polymer i 332. Next, a fifth segment 344 is extruded over the core member 326 and communication lines 328 and is adjacent to and in direct contact with the fourth segment 342. The fifth segment 344 is formed such that the polymer composition of the fifth segment 344 includes more of polymer ii 334 and less of polymer i 332 in comparison to the fourth segment 342. The extrusion of polymer i 332 and polymer ii 334 may occur from a distal portion of the core member 326 to a proximal portion of the core member 326 as described herein.

In some instances, the extrusion of polymer i 332 and polymer ii 334 occurs from a proximal portion of the core member 326 to a distal portion of the core member 326. In some aspects, the extrusion of polymer i 332 occurs prior to the extrusion of polymer ii 334. In other aspects, polymer ii 334 is extruded simultaneously to polymer i 332. In other aspects, polymer i 332 and polymer ii 334 are combined prior to extrusion. Mixing/co-extruding polymer i 332 and polymer ii 334 can form a homogonous mixture with a single durometer between the durometers of the polymer i 332 and the polymer ii 334. The value of the single durometer can depend on the relative amounts or percentages of the polymer i 332 and the polymer ii 334 used in the mixture. In one or more aspects, the communication lines 328 can be wrapped around the core member 326 and the polymer jacket can be applied before and/or after coupling other components together. In some instances, the distal portion 318 of the intraluminal device 316 is formed as a sub-assembly that is then coupled to more proximal portions, such as the proximal portion 109 of FIG. 1.

It is understood that the methods of forming or manufacturing a sensing guide wire in accordance with the present disclosure are exemplary and do not limit the manner in which the devices of the present disclosure can be made. For example, in one or more aspects, the polymer jacket 330 can be formed using any suitable means, including molding, extruding, and/or combinations thereof. In some implementations the core member 326 wrapped with the communication lines 328 can be placed in a mold and the polymers (polymer i 332 and polymer ii 334) can be each injected at varying ratios into the mold to form the multi-durometer polymer jacket 330. In other implementations, the core member 326 with the communication lines 328 is moved relative to a chamber containing polymer i 332 and/or polymer ii 334. For example, the core member 326 with the communication lines 328 can be pulled (or pushed) through the chamber containing the polymer i 332 and/or polymer ii 334 such that the polymers are applied along the length of the core member 326 to define the polymer jacket 330. In some instances, an opening adjacent to the chamber (e.g., an exit of the chamber or a separate component adjacent the exit of the chamber) has an outer diameter 346 approximately equal to the desired outer diameter of the polymer jacket 330. In this regard, as the core member 326 with the polymer applied from the chamber passes through the opening, the excess polymer composition extending beyond the desired diameter can be removed by contact with the boundary of the opening. Alternatively, a first chamber containing the polymer i 332 and a second chamber containing polymer ii 334 each can be moved over the core member 326 with the communication lines 328, while maintaining the core member 326 stationary. The first and second chambers can have a pressurized supply of polymer i 332 and/or polymer ii 334 to ensure that an adequate supply of each polymer is always present for coating the core member 326 to form the polymer jacket 330.

Further, the polymer jacket 330 may eliminate alignment and hinge point issues associated with connections such as where the core member 326 meets the housing 320. The housing 320 may be formed from a rigid material. A kink point may develop when a relatively soft durometer polymer is immediately adjacent to the rigid housing 320. The present disclosure addresses that issue by using a multi-durometer polymer jacket 330, which places a relatively harder polymer immediately adjacent to the rigid housing 320 and transitions to a relatively softer polymer further from the housing 320. The present disclosure provides for a multi-durometer polymer jacket 330 that has a relatively harder polymer at the distal portion of the intravascular device 316. It is unconventional to place a relatively harder polymer at the distal portion of the intravascular device 316 because typically softer materials are provided at the distal portion. In this regard, the polymer jacket 330 may be applied such that it transitions smoothly from a relatively hard polymer composition such as the first segment 336 when adjacent to the housing 320 to a relatively softer polymer composition (such as the second segment 338) adjacent to the relatively hard polymer composition (the first segment 336). The polymer jacket 330 may reduce mechanical and electrical strain at these connections. By forming the polymer jacket 330 directly around the communication lines 328 and core member 326 a strong mechanical coupling is provided along the entire length of the polymer jacket 330, which significantly increases the torque transfer. In some instances, the polymer jacket 330 does not have a constant thickness, but instead conforms to the shape of the communication lines 328 and the core member 326 while maintaining a uniform outer diameter 346, the space available for the core member 326 and the communication lines 329 may be maximized, which may provide improved handling performance by allowing the for the use of larger diameter core members. Finally, the use of a polymer jacket 330 in accordance with the present disclosure can reduce the cost and complexity of manufacturing, as the application of the polymer is reasonably fast, the polymer application system provides alignment of the polymer during the application process, and the cost of the polymer is minimal compared to more traditional coils or tubes that have to be manufactured and then assembled with adhesives.

Segments 336, 338, 340, 342, and/or 344 can be examples of various sections (e.g., a first section, a second section, a third section, etc.) of the polymer jacket 330.

Referring to FIG. 4, shown therein is a diagrammatic, schematic side view of an intravascular device 348 according to an aspect of the present disclosure. The intravascular device 348 includes a distal portion 350 and a proximal portion 351. As shown, the distal portion 350 includes a housing 352 containing component 354. The component 354 is representative of one or more electronic, optical, or electro-optical components. The intravascular device 348 includes a distal tip portion 356. The distal tip portion 356 includes a distal end 358 of the distal portion 350. The proximal portion 351 is defined by a proximal end 359, which opposes the distal end 358. A proximal portion of the core member 360 extends from the proximal portion 351 into the housing 352. A distal portion of the core member 362 extends from the housing 352 into the distal tip portion 356. The distal portion of the core member 362 is coupled to a shaping ribbon 364. The shaping ribbon 364 extends within the distal tip portion 356. A first polymer jacket 366 is disposed proximate to the housing 352 and extends along the distal tip portion 356 of the intravascular device 348. The first polymer jacket 366 extends around at least a portion of the distal portion of the core member 362. A second polymer jacket 368 is disposed proximate to the housing 352 and surrounds at least a portion of the proximal portion of the core member 360.

The first polymer jacket 366 includes a first segment 370 along a first length of the first polymer jacket 366 and includes a polymer composition of at least two polymers. The first segment 370 is located at or near the distal end 358. The first segment 370 includes exemplary composition “A”, which is shown in the legend 372. The legend 372 describes the various polymer compositions “A”, “B”, “C”, “D”, and “E” and provides a relationship between each of the polymer compositions and their relative durometer values. For example, composition “A” is the softest composition relative to compositions “B”, “C”, “D”, and “E”; composition “B” is less hard than composition “C”, composition “C” is less hard than composition “D”, and composition “E” is the hardest composition in comparison to compositions “A”, B″, “C”, and “D”. In some implementations, compositions “A”, “B”, “C”, “D”, and “E” are each comprised of the same type of polymers at varying ratios to change their relative durometer values (such as described in FIGS. 3A-C). The first polymer jacket 366 also includes a second segment 374 along a second length of the first polymer jacket 366 and includes a polymer composition of the at least two polymers. The second segment 374 includes exemplary composition “C”. Therefore, the second segment 374 is harder and more rigid than the first segment 370, in this exemplary aspect. The second segment 374 is disposed between the housing 352 and the first segment 370. The second segment 374 is in direct contact with the housing 352. In order to have a smooth transition from the harder second segment 374 to the softer first segment 370, a transition region 376 is included. The transition region 376 includes a polymer composition of the at least two polymers. The transition region 376 includes a polymer composition that has a greater durometer value than the first segment 370 and a lesser durometer value than the second segment 374 to provide a smooth transition between the first and second segments 370, 374.

The second polymer jacket 368 includes a first segment 378 along a first length of the second polymer jacket 368 and includes a polymer composition of at least two polymers. The first segment 378 includes exemplary composition “D”. The first segment 378 is in direct contact with the housing 352. The second polymer jacket 368 includes a second segment 380 along a second length of the second polymer jacket 368 and includes a polymer composition of at least two polymers. The second segment 380 includes exemplary composition “B”. The first segment 378 along the first length and the second segment 380 along the second length are separated by the transition region 382. The transition region 382 includes a polymer composition of the at least two polymers. The transition region 382 includes a polymer composition that has a greater durometer value than the second segment 380 and a lesser durometer value than the first segment 378 to provide a smooth transition between the first and second segments 378, 380. The second polymer jacket 368 also includes a third segment 384 along a third length of the second polymer jacket 368 and includes a polymer composition of the at least two polymers. The third segment 384 includes exemplar composition “E”. A transition region 386 extends between the second segment 380 and the third segment 384. The transition region 386 includes a polymer composition of the at least two polymers. The transition region 386 includes a polymer composition that has a greater durometer value than the second segment 380 and a lesser durometer value than the third segment 384.

The component 354 is representative of one or more electronic, optical, or electro-optical components. In some instances, the component 354 is positioned less than 10 cm, less than 5, or less than 3 cm from the distal end 358. In that regard, the component 354 is a pressure sensor, a flow sensor, a temperature sensor, an imaging element, an optical fiber, an ultrasound transducer, a reflector, a mirror, a prism, an ablation element, an RF electrode, a conductor, and/or combinations thereof. The specific type of component or combination of components can be selected based on an intended use of the intravascular device. In some instances, the component 354 is positioned within the housing 352. In some aspects, communication lines, as described herein, are included on the intravascular device 348.

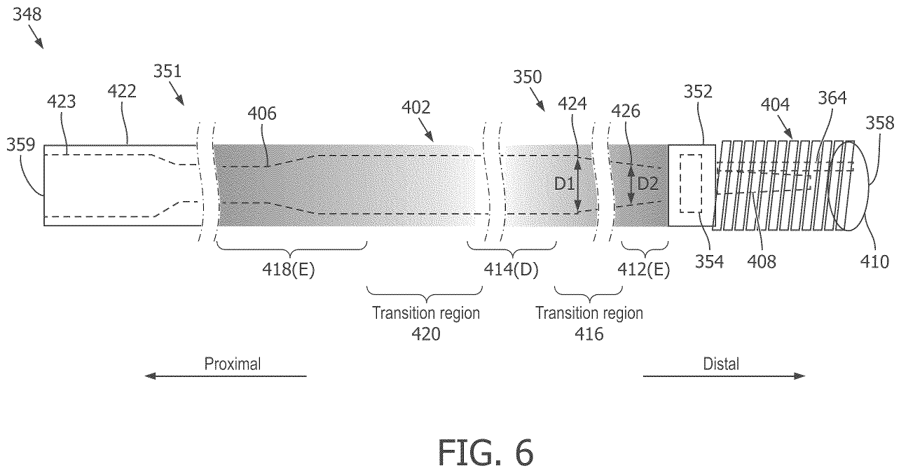

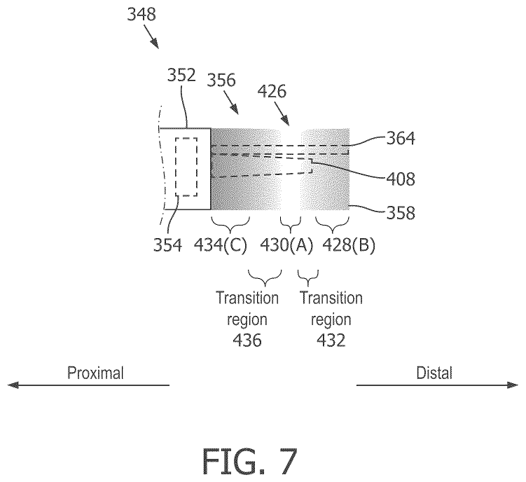

FIG. 4 may illustrate a distal portion 350 and proximal portion 351 of an intravascular device 348 (e.g., intravascular guidewire), such as the distal portion 107 and the proximal portion 109 of the intravascular device 102, which is depicted as a pressure-sensing guidewire in FIG. 1, the distal portion 350 and the proximal portion 351 of the intravascular device 348, which is shown as a pressure-sensing guidewire depicted in FIG. 6, and the distal tip portion 356 of the intravascular device 348, which is depicted as a pressure-sensing guidewire in FIG. 7. The intravascular device 348 (e.g., intravascular guidewire) of FIG. 4 can include other features of the intravascular device 102 in FIG. 1 and the intravascular device 348 in FIG. 6 and/or in FIG. 7. In some aspects, the intravascular device 348 is an intraluminal device. In one or more aspects, the intravascular device 348 is a guide wire such as a pressure-sensing guidewire

In some aspects, the polymer composition of “A”, “B”, “C”, “D”, and “E” are each composed of varying ratios of polymer i 332 and polymer ii 334, shown in FIGS. 3A-3C, such that ratio of the polymers are as follows: “A” is (1%<<ii %), “B” is (1%<ii %), “C” is (1%≈ii %), “D” is (i %>ii %), and “E” is (1%>>ii %). In some aspects, the polymer composition of “A”, “B”, “C”, “D”, and “E” are the same two polymers at varying ratios, which changes the durometer values of the compositions. In other aspects, one or more of the polymer compositions of “A”, “B”, “C”, “D”, and “E” include at least one type of polymer that is different from the remainder of polymer compositions. In some aspects, polymer composition “A” only includes a single polymer such as polymer ii 334. In one or more aspects, polymer composition “E” only includes a single polymer i 332. In other compositions, polymer composition “A” and polymer composition “E” includes both polymer i 332 and polymer ii 334 at varying ratios. In at least one aspect, the polymer compositions “A”, “B”, “C”, “D”, and “E” represent threshold percentage-range values. For example, polymer composition “A” may be 0-20% of polymer i 332 and 80-100% of polymer ii 334 and polymer composition “E” may be 80-100% of polymer i 332 and 0-20% of polymer ii 334. In some instances, the relationship between the first segment 378, the second segment 380, and the transition region 382 of the second polymer jacket 368 is similar to FIG. 3C, showing a smooth transition between two compositions, such as polymer i 332 and polymer ii 334. That is, all of FIG. 3C may be shown between only two compositions in FIG. 4 (such as first segment 378 and second segment 380 or second segment 380 and third segment 384), and the remainder of the compositions in FIG. 4 may differ. In some instances, each longitudinal location along the intravascular device 348 has a uniform material composition. For example, around the circumference at a first longitudinal location, the material composition is the same (e.g., a first composition or combination of two or more polymers such as polymer i 332 and polymer ii 334), and at a different, second longitudinal location, the material composition (e.g., a second composition/combination of two or more polymers) is different than the first longitudinal location.

In some instances, proximate indicates that two objects are directly adjacent to one another or in direct contact with one another. In other instances, proximate indicates that two objects are adjacent to one another and are spaced apart via a transition region such as transition region 376. For instance, transition region 376 may be in between the second segment 374 and the first segment 370, but the second segment 374 is proximate to the first segment 370. In several aspects, the second segment 374 of the first polymer jacket 366 is proximate to the housing 352. For example, the second segment 374 may be proximate to the first segment 370 and be directly in contact with the first segment 370, in some instances. In other instances, the second segment 374 is proximate to the first segment 370 and is separated from the first segment by transition region 376. In several aspects, the first segment 378 of the second polymer jacket 368 is proximate to the housing 352. In several aspects, the first segment 378 of the second polymer jacket 368 is proximate to the second segment 380, the transition region 382, and the sensor housing 352. The second segment 380, in various aspects, is proximate to the first segment 378, the third segment 384, and the transition regions 382, 386. In various aspects, the third segment 384 may be proximate to the second segment 380 and/or the transition region 386.

The change in hardness/material composition changes along the length of device. In some aspects, the hardness/material composition does not change along around the circumference

In some aspects, the first polymer jacket 366 and the second polymer jacket 368 are composed of the same types of polymers, but at different ratios. In other aspects, the first polymer jacket 366 includes at least one differing polymer from the second polymer jacket 368. In some instances, the polymers of the polymer compositions are the polymers described herein in FIGS. 3A-3C.

In at least one aspect, the relative durometer values, shown in legend 372, are based on the durometer shore hardness scale. Example ranges of the durometer values can include between 30A and 85A, between 45A and 55A, between 45A and 80A, between 65A and 80A, and/or other ranges, including values such as 50A, 70A, 75A, and/or other values both larger and smaller. Each polymer composition (A-E) can have a respective durometer value within these example ranges. In several aspects, the relative durometer values, shown in legend 372, are based on the relative hardness of one of the polymer compositions in comparison to another polymer composition shown in the legend 372. In some aspects, durometer means level of hardness, rigidity, and/or resistance to breaking, bending, and/or indentation. Higher values indicate a material that has greater hardness, rigidity, and/or a greater resistance to breaking, bending, and/or indentation, whereas lower values indicate a material that is more flexible, less rigid, and a lower resistance to breaking, bending, and/or indication.

In some instances, first polymer jacket 366 includes one or more additional segments in between the first segment 370 and the second segment 374. In several instances, the first polymer jacket 366 is distal (e.g., immediately adjacent and in direct contact with) a proximal end of the rigid sensor housing 352, opposite to a distal end. In some aspects, the second segment 374 is a different polymer composition with a greater durometer value such as polymer composition D. In one or more aspects, the transition region 376 and/or the first segment 370 are a greater length of the distal tip portion 356 than the second segment 374. The distal tip portion 356 may extend from one side of the housing 352 to the distal end 358. The distal tip portion 356, in some instances, is 3 cm in length. In other aspects, the distal tip portion 356 is 2 cm to 5 cm in length. In some aspects the second segment 374 of the first polymer jacket 366 is the same composition and/or length as the first segment 378 of the second polymer jacket 368. In some instances, the first polymer jacket 366 includes more than two polymers. In some aspects, the first polymer jacket 366 includes the same polymers as the second polymer jacket 368.

In some instances, the second polymer jacket 368 includes more than two polymers. In several instances, the second polymer jacket 368 is proximate (e.g., immediately adjacent and in direct contact with) a proximate end of the rigid housing 352. In other instances, the second polymer jacket 368 includes only two polymers; however, different segments (such as the first segment 378 and the second segment 380) have different ratios of the first polymer and the second polymer. The first and second polymer may be the polymer i 332 and polymer ii 334 described herein. In some aspects, the transition regions such as transition region 382 comprise varying ratios of the polymers over the length of the transition region in order to change the polymer composition from one segment to the next such as the first segment 378, which may be a “D” polymer composition to the second segment 380, which may be a B polymer composition. The transition regions 382, 386 and the first, second, and third segments 378, 380, 384 may be of varying length along the intravascular device 148. The intravascular device 348 may have a working length of approximately 150-200 cm in length, in accordance with some aspects. In some aspects, the working length is 185 cm. In at least one aspect, the intravascular device 348 includes a flexible length that is approximately 40 cm in length. In some aspects, the first polymer jacket 366 and the second polymer jacket 368 are one polymer jacket that extends around the entirety of the flexible length of the intravascular device 348. In other aspects, the first polymer jacket 366 extends around the distal tip portion 356 and the second polymer jacket 368 extends around the remainder of the flexible length of the intravascular device 348 except for at the housing 352 (as shown in FIG. 4). In some instances, the third segment 384 is the longest and hardest segment, as the stiffness is needed to push and/or maneuver the intravascular device 348. In some instances, the third segment 384 has the greatest relative durometer in comparison to the first segment 378 and the second segment 380. In several aspects, the third segment 384 has an “E” polymer composition, while the second segment 380 has a “B” polymer composition, and while the first segment 378 has a “D” polymer composition. In some instances, the relative durometer values of the second polymer jacket 368 is a greater durometer value at the first segment 378, a lesser durometer value at the second segment 380 (in comparison to either the first segment 378 or the third segment 384), and a greater durometer value at the third segment 384 in comparison to the second segment 380. In one or more aspects, the first segment 378 has the same relative durometer value as the third segment 384.

In various aspects, a solder ball or other suitable element is secured to the distal end 358. In one or more aspects, the solder ball defines the distal tip portion 356 of the intravascular device 348 with an atraumatic tip suitable for advancement through patient vessels, such as vasculature. In some instances, a flow sensor is positioned at the distal tip portion 356 instead of the solder ball.

In some implementations, the proximal portion of the core member 360 and the distal portion of the core member 362 are an integral component (i.e., the proximal portion of the core member 360 extends through the housing 352 to define distal portion of the core member 362). Generally, the proximal portion of the core member 360 and the distal portion of the core member 362 are sized, shaped, and/or formed out of particular material(s) to create a desired mechanical performance for the distal portion 350 of the intravascular device 348. In some particular implementations, the distal portion of the core member 362 is coupled to the shaping ribbon 364.

Segments 370, 374, 378, 380, and/or 384, and/or the regions 376, 382, and/or 386, can be examples of various sections (e.g., a first section, a second section, a third section, etc.) of a polymer jacket (e.g., the polymer jacket 366 or the polymer jacket 368).

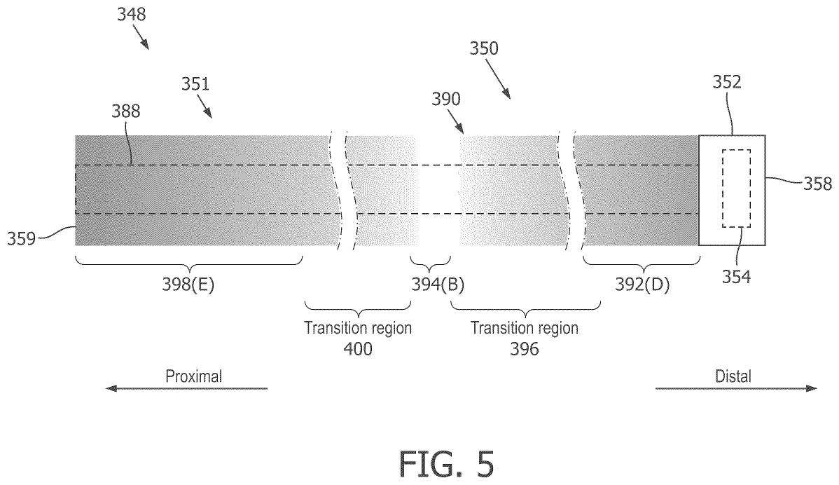

Referring now to FIG. 5, another aspect of the intravascular device 348 is shown and includes several components of FIG. 4, which components are given the same reference number. The intravascular device 348 includes the distal portion 350 and the proximal portion 351. The distal portion 350 of the intravascular device 348 includes housing 352 containing component 354. The component 354 is representative of one or more electronic, optical, or electro-optical components. A distal end 358 of the intravascular device 348 is located at the housing 352. The distal portion 350 terminates at the distal end, and the proximal portion 351 terminates at a proximal end 359, which opposes the distal end 358. The intravascular device 348 includes a core member 388 that from the proximal portion (not shown) into the housing 352. A polymer jacket 390 is disposed adjacent to the housing 352 and surrounds at least a portion of the core member 388. The polymer jacket 390 includes a first segment 392 along a first length of the polymer jacket 390 and includes a polymer composition of at least two polymers. The first segment 392 is positioned adjacent to the housing 352. The first segment 392 includes exemplary composition “D,” which is shown in the legend 372 on FIG. 4. The polymer jacket 390 also includes a second segment 394 along a second length of the polymer jacket 390 and includes a polymer composition of at least two polymers. The second segment 394 includes exemplary composition “B”. Therefore, the second segment 394 is less rigid than the first segment 392. A first transition region 396 extends between the first segment 392 and the second segment 394. The first transition region 396 provides a smooth transition from the rigid first segment 392 to the less rigid second segment 394. The first transition region 396 includes a polymer composition of the at least two polymers. The first transition region 396 includes a polymer composition that has a lesser durometer value than the first segment 392 and a greater durometer value than the second segment 394 to provide a smooth transition between the first and second segments 392, 394. The polymer jacket 390 also includes a third segment 398 along a third length of the polymer jacket 390 and includes a polymer composition of at least two polymers. The third segment 398 includes exemplary composition “E”. Therefore, the third segment 398 is more rigid than the first segment 392 and more rigid than the second segment 394. A second transition region 400 extends between the second segment 394 and the third segment 398. The second transition region 400 provides a smooth transition from the rigid third segment 398 to the less rigid second segment 394. The second transition region 400 includes a polymer composition of the at least two polymers. The second transition region 400 includes a polymer composition that has a lesser durometer value than the third segment 398 and a greater durometer value than the second segment 394 to provide a smooth transition between the second and third segments 394, 398.

FIG. 5 may illustrate the distal portion 350 and proximal portion 351 of an intravascular device 348 (e.g., intravascular guidewire), the distal portion 107 and the proximal portion 109 of the intravascular device 102 depicted as a flow sensing guidewire in FIG. 2. The intravascular device 348 (e.g., intravascular guidewire) of FIG. 5 may include other features of the intravascular device 102 in FIG. 2.

In some instances, component 354 is a flow sensor. In some instances, the core member 388 includes a non-varying or constant diameter along the length of the intravascular device 348. In various aspects, communication lines (as described herein) are wrapped around and/or extend along the length of the core member 388 and couple to a portion of the housing 352 and/or component 354.

In at least one aspect, the first segment 392 has the same polymer composition as the third segment 398. In various aspects, the third length (the length of the third segment 398) is the longest of the segments and/or the first and the second transition regions 396, 400. In some aspects, the first length (the length of the first segment 392) is the shortest of the segments and/or the first and the second transition regions 396, 400. In several aspects, the second segment 394 is a different polymer composition than “B”, but the polymer composition includes a relative durometer value that is less than either the first segment 392 and the third segment 398 such as an “A” or “C” polymer composition. In one or more aspects, the first transition region 396 and the second transition region 400 include varying polymer compositions along their respective lengths.

Segments 392, 394, 398, and/or the regions 396 and/or 400, can be examples of various sections (e.g., a first section, a second section, a third section, etc.) of the polymer jacket 390.

Referring now to FIG. 6, shown therein is a diagrammatic, schematic side view of the intravascular device 348, according to an aspect of the present disclosure, and includes several components of FIGS. 4-5, which components are given the same reference numbers. The intravascular device 348 includes a distal portion 350 and a proximal portion 351. As shown, the distal portion 350 includes a more proximal flexible element 402 and a more distal flexible element 404 on each side of a housing 352 containing component 354. The more proximal portion of the core member 406 extends through the more proximal flexible element 402. The proximal flexible element can be a polymer jacket surrounding the core member 406. Similarly, a more distal portion of the core member 408 extends through the more distal flexible element 404. In some implementations, the proximal portion of core member 406 and the distal portion of core member 408 are an integral component (i.e., the proximal portion of core member 406 extends through the housing 352 to define distal portion of core member 408). In some instances, the distal portion of core member 408 is coupled to the shaping ribbon 364. The shaping ribbon 364 can be coupled to various components of the intravascular device 348, including the housing 352 and/or adhesive within and/or surround the housing 352, the distal portion of core member 408, and/or the distal end 358 using adhesives, solder, mechanical coupling, and/or combinations thereof. Generally, the proximal portion of core member 406, the distal portion of core member 408, and the shaping ribbon 364 are sized, shaped, and/or formed out of particular material(s) to create a desired mechanical performance for the distal portion 350 of the intravascular device 348. For example, the proximal portion of core member 406, the distal portion of core member 408, and the shaping ribbon 364 can be formed from a flexible and/or elastic material, including metals or metal alloys such as nickel titanium or nitinol, nickel titanium cobalt, stainless steel, and/or various stainless-steel alloys. In some particular implementations, the proximal portion of core member 406 and the distal portion of core member 408 are formed of nitinol and the shaping ribbon 364 is formed of stainless steel. However, any combination of materials can be used in accordance with the present disclosure.

The proximal flexible element 402 may be a polymer jacket. The polymer jacket may be composed of two or more types of polymers at varying ratios as described herein. The distal flexible element 404 can be any suitable flexible element, including polymer tubes, coils, and/or coil-embedded polymer tubes. In the illustrated aspect the proximal flexible element 402 is a polymer jacket and the distal flexible element 404 is a coil. In other aspects, the distal flexible element 404 is also a polymer jacket. The proximal and/or distal flexible elements 402, 404 can be at least partially filled with two or more polymers to improve the mechanical performance and durability of the intravascular device 348. In that regard, in some instances at least two polymers with varying degrees of durometer are utilized to provide a desired transition in bending stiffness along the length of the intravascular device 348, as described herein. A solder ball 410 or another suitable element is secured to the distal end 358. As shown, the solder ball 410 defines the distal end 358 of the intravascular device 348 with an atraumatic tip suitable for advancement through patient vessels, such as vasculature. In some aspects, a flow sensor is positioned at the distal end 358 instead of the solder ball 410.

The proximal flexible element 402 includes a first segment 412 along a first length of the proximal flexible element 402 and may include a polymer composition of at least two polymers. The first segment 412 is positioned adjacent to the housing 352. The first segment 412 includes exemplary composition “E,” which is shown in the legend 372 on FIG. 4. In some implementations, compositions “A”, “B”, “C”, “D”, and “E” are each comprised of the same type of polymers at varying ratios to change their relative durometer values (such as described in FIGS. 3A-C). The proximal flexible element 402 also includes a second segment 414 along a second length of the proximal flexible element 402 and may include a polymer composition of at least two polymers. The second segment 414 includes exemplary composition “D”. Therefore, the second segment 414 is less rigid than the first segment 412. A first transition region 416 extends between the first segment 412 and the second segment 414. The first transition region 416 provides a smooth transition from the rigid first segment 412 to the less rigid second segment 414. The first transition region 416 includes a polymer composition of the at least two polymers. The first transition region 416 includes a polymer composition that has a lesser durometer value than the first segment 412 and a greater durometer value than the second segment 414 to provide a smooth transition between the first and second segments 412, 414. The proximal flexible element 402 also includes a third segment 418 along a third length of the proximal flexible element 402 and may include a polymer composition of at least two polymers. The third segment 418 includes exemplary composition “E”. Therefore, the third segment 418 is the same rigidity as the first segment 412 and more rigid than the second segment 414. A second transition region 420 extends between the second segment 414 and the third segment 418. The second transition region 420 provides a smooth transition from the rigid third segment 418 to the less rigid second segment 414. The second transition region 420 includes a polymer composition of the at least two polymers. The second transition region 420 includes a polymer composition that has a lesser durometer value than the third segment 418 and a greater durometer value than the second segment 414 to provide a smooth transition between the second and third segments 414, 418.

The intravascular device 348 includes a proximal subassembly 422 adjacent to the third segment 418 along the proximal end of the intravascular device 348. The proximal subassembly 422 includes a core member 423. In various instances, the core member 423 is distinct from the proximal portion of the core member 406. In several aspects, the core member 423 is made of a different material than the proximal portion of the core member 406 and/or the distal portion of the core member 408. In some aspects, the proximal subassembly 422 is 145 cm in length. In one or more aspects, the proximal subassembly 422 is 130-155 cm in length. The proximal subassembly 422 is further defined by the proximal end 359, which opposes the distal end 358.

The proximal portion of core member 406 and the distal portion of the core member 408 each include varying diameters along the length of the intravascular device 348. The varying diameters are perpendicular to the length of the intravascular device 348. The proximal portion of the core member 406 includes a first diameter “D1” 424 and a second diameter “D2” 426. The D1 424 is greater than D2 426. The D2 426 is closer to the housing 352 than D1 424. In some instances, the proximal portion of the core member 406 tapers to a smaller diameter such as D2 426 when the core member nears the housing 352 (as shown in FIG. 6). In one or more aspects, the proximal portion of the core member 406 also tapers to a smaller diameter as it approaches the proximal subassembly 422. The proximal portion of the core member 406, in some aspects, has a constant diameter in between the two tapering sections (as shown in FIG. 6). In other aspects, the proximal portion of the core member 406 is a constant diameter along the length of the intravascular device 348. In some instances, the distal portion of the core member 408 also tapers to an even smaller diameter than D2 426. The distal portion of the core member 408, in one or more aspects, has the smallest diameter at a tip of the core member 408, proximate the distal end 358. In some aspects, the distal portion of the core member 408 includes a taper that appears like a step-function having regions of constant slope coupled to regions of infinite slope, where each region of constant slope corresponds to a decrease in diameter from the housing 352 (largest diameter) to the tip of the distal portion of the core member 408 (smallest diameter). In other aspects, the tapering of the proximal portion of the core member 406 and distal portion of the core member 408 may be altered to change the desired support profile. In various instances, the proximal portion of the core member 406 and the distal portion of the core member 408 include varying diameters which may impact the stiffness of the device and/or create a hinge point on the intravascular device 348. In some instances, a higher durometer composition may be selected to offset the change in geometry of the proximal portion of the core member 406 and the distal portion of the core member 408 to provide strain relief at a hinge point of the intravascular device 348. In one or more instances, a higher durometer composition may be selected at a region of infinite slope in comparison to the region of constant slope, in order to change the support profile of the intravascular device 348. For example, in some aspects, a support profile that produce a linear or near linear graph is desired. In some aspects, the distal portion of the core member 408 is coupled to the shaping ribbon 364 using adhesive(s) and at least one connecting sleeve. Further, the connecting sleeves can be formed from any suitable material, including polymers, such as polyimide, pebax, nylon, polyethylene, etc.

Aspects of the present disclosure may include features described in U.S. application Ser. No. 15/745,534, filed Jan. 1, 2018, entitled “Intravascular Devices Systems and Methods with an Adhesively Attached Shaping Ribbon,” the entirety of which is hereby incorporated by reference herein.

FIG. 6 may illustrate a distal portion 350 and proximal portion 351 of an intravascular device 348 (e.g., intravascular guidewire), such as the distal portion 107 and the proximal portion 109 of the intravascular device 102 such as the pressure-sensing guidewire depicted in FIG. 1, the distal portion 350 and the proximal portion 351 of the intravascular device 348 depicted as a pressure-sensing guidewire in FIG. 4, and the distal tip portion 356 of the pressure-sensing guidewire in FIG. 7. The intravascular device 348 (e.g., intravascular guidewire) of FIG. 6 can include other features of the intravascular device 102 in FIG. 1 and/or the intravascular device 348 depicted in FIG. 4, and/or in FIG. 7.

In some implementations, the proximal flexible element 402 extends only a portion of the length of the proximal portion of the core member 406 positioned proximal of the component 354. For example, FIG. 6 shows an aspect where the proximal flexible element 402 extends only a portion of the length of the proximal portion of the core member 406 such that a proximal subassembly 422, which includes a portion of the proximal portion of the core member 406 is exposed. In such instances, the proximal flexible element 402 may be formed to have the desired length to expose the proximal subassembly 422 or a portion of the polymer initially applied to the proximal portion of the core member 406 may be removed to expose the proximal subassembly 422. In this regard, it may be desirable to have the proximal flexible element 402 extend along only a portion of the proximal portion of the core member 406 to facilitate coupling to more proximal portions of the intravascular device 348 (e.g., a guide wire), such as flexible element and/or a proximal section. A similar approach may be utilized to facilitate coupling to distal portions of the intravascular device 348 (such as a guide wire) as well (e.g., in aspects where the communication lines 328 are coupled to the component 354 after forming the polymer jacket 330). In some instances, gap(s) between the proximal flexible element 402 and the adjacent components of the intravascular device 348 are subsequently filled with a polymer, adhesive, and/or other suitable material(s) after coupling the proximal portion of the core member 406 and/or the communication lines 328 to the adjacent proximal and/or distal component(s). In some particular instances, the gap(s) are filled with the same material(s) utilized to form the proximal flexible element 402.

Segments 412, 414, 418, and/or the regions 416 and/or 420, can be examples of various sections (e.g., a first section, a second section, a third section, etc.) of the polymer jacket of the more proximal flexible element 402.

Referring now to FIG. 7, shown therein is a diagrammatic, schematic side view of the housing 352 and the distal tip portion 356 of the intravascular device 348, according to an aspect of the present disclosure, and includes several components of FIGS. 4-6, which components are given the same reference number. As shown, the housing 352 includes component 354. The distal portion of the core member 408 extends through a polymer jacket 426. The distal portion of the core member 408 is coupled to the shaping ribbon 364. The shaping ribbon 364 can be coupled to various components of the intravascular device 348, including the housing 352 and/or adhesive within and/or surround the housing 352, the distal portion of the core member 408, and/or the distal end 358 using adhesives, solder, mechanical coupling, and/or combinations thereof. Generally, the distal portion of the core member 408 and the shaping ribbon 364 are sized, shaped, and/or formed out of particular material(s) to create a desired mechanical performance for the distal portion 350 of the intravascular device 348. For example, the distal portion of the core member 408 and the shaping ribbon 364 can be formed from a flexible and/or clastic material, including metals or metal alloys such as nickel titanium or nitinol, nickel titanium cobalt, stainless steel, and/or various stainless-steel alloys. In some particular implementations, the distal portion of the core member 408 is formed of nitinol and the shaping ribbon 364 is formed of stainless steel. However, any combination of materials can be used in accordance with the present disclosure. The polymer jacket 426 is in direct contact with and adjacent to the housing 352. The polymer jacket 426 extends from the housing 352 to the distal end 358.

The polymer jacket 426 includes a first segment 428 extending from the distal end 358 along a first length of the intravascular device 348 and may include a polymer composition of at least two polymers. The first segment 428 includes exemplary composition “B,” which is shown in the legend 372 on FIG. 4. In some implementations, compositions “A”, “B”, “C”, “D”, and “E” are each comprised of the same type of polymers at varying ratios to change their relative durometer values (such as described in FIGS. 3A-C). The polymer jacket 426 also includes a second segment 430 along a second length of the intravascular device 348 and may include a polymer composition of at least two polymers. The second segment 430 includes exemplary composition “A”. Therefore, the second segment 430 is less rigid than the first segment 428. A first transition region 432 extends between the first segment 428 and the second segment 430. The first transition region 432 provides a smooth transition from the more rigid first segment 428 to the less rigid second segment 430. The first transition region 432 includes a polymer composition of the at least two polymers. The first transition region 432 includes a polymer composition that has a lesser durometer value than the first segment 428 and a greater durometer value than the second segment 430 to provide a smooth transition between the first and second segments 428, 430. The polymer jacket 426 also includes a third segment 434 along a third length of intravascular device 348 and may include a polymer composition of at least two polymers. The third segment 434 includes exemplary composition “C”. Therefore, the third segment 434 is the more rigid than the first segment 428 and more rigid than the second segment 430. A second transition region 436 extends between the second segment 430 and the third segment 434. The second transition region 436 provides a smooth transition from the rigid third segment 434 to the less rigid second segment 430. The second transition region 436 includes a polymer composition of the at least two polymers. The second transition region 436 includes a polymer composition that has a lesser durometer value than the third segment 434 and a greater durometer value than the second segment 430 to provide a smooth transition between the second and third segments 430, 434.

FIG. 7 may illustrate a distal tip portion 356 of an intravascular device 348 (e.g., intravascular guidewire), such as the distal end 108 of the intravascular device 102 depicted as a pressure-sensing guidewire in FIG. 1, the distal tip portion 356 of the intravascular device shown as a pressure-sensing guidewire in FIG. 4, and the distal tip portion 356 of the intravascular device 348 depicted as a pressure-sensing guidewire in FIG. 6. The intravascular device 348 (e.g., intravascular guidewire) of FIG. 7 can include other features of the intravascular device 102 in FIG. 1, intravascular device 348 in FIG. 4, and/or intravascular device 348 in FIG. 6. For example, in some aspects, the polymer jacket 426 corresponds to the distal flexible element 404 of FIG. 6.

Segments 428, 430, 434, and/or the regions 432 and/or 436, can be examples of various sections (e.g., a first section, a second section, a third section, etc.) of the polymer jacket 426.

The logical operations making up the aspects of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use electrical and electronic devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the metal ink conductor assembly. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
an intravascular guidewire configured to be positioned within a blood vessel and comprising:
a proximal portion and a distal portion;
a core wire;
a polymer jacket positioned around the core wire;
a sensor housing positioned at the distal portion; and
a sensor positioned within the sensor housing and configured to obtain medical data associated with the blood vessel,
wherein the polymer jacket comprises a first section with a first hardness and a second section with a second hardness.

2. The apparatus of claim 1, wherein the first hardness is less than the second hardness.

3. The apparatus of claim 1,
wherein the first hardness is larger than the second hardness,
wherein the first section is proximate to the sensor housing, and
wherein the second section is proximate to the first section.

4. The apparatus of claim 3,
wherein the first section comprises a first composition of at least two polymers, and
wherein the second section comprises a second composition of the at least two polymers.

5. The apparatus of claim 3,
wherein the polymer jacket comprises a third section with a third hardness,
wherein the third section is proximate to the second section such that the second section is disposed directly between the first section and the third section, and
wherein the third section comprises a third composition of the at least two polymers.

6. The apparatus of claim 5, wherein the third hardness is larger than both the first hardness and the second hardness.

7. The apparatus of claim 3,
wherein the sensor housing comprises a first proximal end and an opposing distal end,
wherein the distal end is distal of the proximal end, and
wherein the polymer jacket is in direct contact with the proximal end of the sensor housing.

8. The apparatus of claim 3,
further comprising a further polymer jacket comprising a third section with a third hardness and a fourth section with a fourth hardness,
wherein the third hardness is larger than the fourth hardness,
wherein the fourth section is distal of the third section, and
wherein the third section is proximate to the distal end of the sensor housing.

9. The apparatus of claim 8,
wherein the sensor housing comprises a first proximal end and an opposing distal end,
wherein the distal end is distal of the proximal end,
wherein the polymer jacket is in direct contact with the proximal end of the sensor housing, and
wherein the further polymer jacket is in direct contact with the distal end of the sensor housing.

10. An apparatus, comprising:
an intravascular guidewire configured to be positioned within a blood vessel and comprising:
a proximal portion and a distal portion;
a core wire;
a polymer jacket positioned around the core wire;
a sensor housing positioned at the distal portion; and
a sensor positioned within the sensor housing and configured to obtain medical data associated with the blood vessel,
wherein the polymer jacket comprises:
a first section comprising a first composition of at least two polymers;
a second section comprising a second composition of the at least two polymers; and
a third section comprising a third composition of the at least two polymers and located between the first section and the second section,
wherein the third composition comprises a transition between the first composition and the second composition.

11. The apparatus of claim 10,
wherein the first section comprises a first hardness, and
wherein the second section comprises a second hardness different than first hardness.

12. The apparatus of claim 11, wherein the first hardness is greater than the second hardness.

13. The apparatus of claim 10,
wherein the sensor housing is located at a distal end of the distal portion, and
wherein the distal portion terminates at the distal end.

14. The apparatus of claim 13, wherein the first section of the polymer jacket is in direct contact with the sensor housing.

15. The apparatus of claim 10, wherein the first composition, the second composition, and the third composition each comprise different percentages of the at least two polymers.

16. The apparatus of claim 15,
wherein the polymer jacket is disposed proximal of the sensor housing, and
wherein the second section is proximal of the first section and proximal of the third section.

17. The apparatus of claim 16, further comprising:
a further polymer jacket disposed distal of the sensor housing and positioned around the core wire.

18. The apparatus of claim 15,
wherein the polymer jacket is disposed distal of the sensor housing,
wherein the second section is distal of the third section, and
wherein the third section is distal of the first section.

19. An apparatus, comprising:
an intravascular guidewire configured to be positioned within a blood vessel and comprising:
a proximal portion and a distal portion;
a core wire;
a polymer jacket positioned around a length of the core wire;
a sensor housing positioned at the distal portion; and
a sensor positioned within the sensor housing and configured to obtain medical data associated with the blood vessel,
wherein the sensor housing is relatively more rigid than the polymer jacket,
wherein the polymer jacket comprises varying durometers along the length,
wherein the polymer jacket comprises a first section and a second section,
wherein the first section is disposed directly adjacent to the sensor housing, and
wherein the first section has a greater durometer than the second section.

20. The apparatus of claim 19, wherein the second section is located proximal of the first section.

21. The apparatus of claim 19, wherein the second section is located distal of the first section.
